(19) United States
(12) Patent Application Publication
(10) Pub. No.: US 2014/0274886 A1
(43) Pub. Date: Sep. 18, 2014

(54) METHODS AND SYSTEMS FOR ADJUSTING RUMEN UNDEGRADED PROTEIN IN ANIMAL DIETS

(71) Applicant: Forage Genetics International, LLC, West Salem, WI (US)

(72) Inventor: David C. Weakley, Pacific, MO (US)

(21) Appl. No.: 13/828,597

(22) Filed: Mar. 14, 2013

(51) Int. Cl. A23K 1/16; A61K 38/16; A23K 1/18

(52) U.S. Cl. CPC A23K 1/1631; A23K 1/1813; A61K 38/168
USPC 514/4.7; 426/2; 426/231; 514/1.1

(57) ABSTRACT

In a ruminant feed, rumen undegraded protein (“RUP”), which is protein orally ingested and passed through the rumen in an undegraded or undigested state, is determined through analysis of feed components in the feed and is used to modify the feed formulation to control a level of RUP exiting the rumen of the ruminant. Additionally the RUP and rumen undegraded neutral detergent fiber (“RUNDF”) content of selected feed components may be received based on an analysis of the feed components and the feed ration may be formulated accordingly. The RUP content may be determined by comparing analysis results with a calibration curve of previously analyzed feed components.

### FIELD OF THE INVENTION

The present disclosure relates to methods and systems for determining an amount of protein in a feed ration, and more particularly relates to determining an amount of rumen undegraded protein in a feed ration for reducing feed costs, improving milk production, improving meat production and/or reducing nitrogen excretion.

### BACKGROUND

Dietary protein provides a source of amino acids that may be converted into milk protein in lactating ruminants. A portion of the dietary protein is soluble in the rumen and is degraded by microorganisms in the ruminal fluid into amino acids and ammonia. In addition, amino acids are produced in the rumen through the assimilation of non-protein nitrogen (e.g., ammonia) and available amino acids and peptides by ruminal microbes, which synthesize microbial protein. This microbial protein subsequently passes into the small intestine and the digested amino acids are absorbed through the wall of the small intestine and used by the animal for milk production. In addition to the microbial protein flowing into the small intestine to provide a supply of absorbable amino acids, dietary protein escaping the rumen undigested or undegraded, known as rumen undegraded protein (“RUP”) flows into the small intestines, as well, to provide a source of amino acids. The combination of protein sources such as RUP, microbial proteins and endogenous protein absorbed by the small intestine is known as metabolizable protein (“MP”).

Determining MP in the diet is challenging because typically MP is calculated, in part, based on crude protein in the feed ration, and nutritionists assume that the amount of protein and RUP in protein sources are fixed, such as a high protein ingredient having a crude protein content of 50%, but only half of the protein is ruminally undegraded. In this case the high protein ingredient has a RUP content of only 25%. However, the level of protein and the level of RUP in feed components varies according to protein source (e.g., alfalfa hay and soybean meal), and is variable within a given protein source.

### SUMMARY

Implementations determine rumen undegraded protein (“RUP”) levels in feed components in animal diets and the RUP levels are utilized in formulating a feed ration to reach a target RUP level to improve performance (e.g., milk and/or meat production improvement and/or nitrogen excretion reduction) and/or reduce feed costs. According to the present disclosure, the RUP in the animal diet is controlled by accurately calculating the amount of RUP in feed components, as opposed to deducing RUP from assumed tabular values, and formulating a feed ration to deliver a target amount of RUP in the diet based on the calculated RUP amounts.

In one implementation, a method for improving performance of a ruminant and/or reducing feed costs involves receiving a level of RUP in relation to dry matter in a ruminant feed ration. The RUP level is a portion of protein in the feed ration to be orally ingested and passed through the rumen in an undegraded state, and is based on an analysis of RUP present in one or more feed components in the feed ration. Using the received RUP level, the feed ration is modified to control an amount of RUP exiting the rumen. The modified feed ration may be fed to the ruminant to improve performance and/or reduce feed costs.

In another implementation, a method for formulating a ruminant feed involves selecting feed components for use in a ruminant feed ration and receiving nutritional composition analysis results for the selected feed components. The results include a RUP content and a rumen undegraded neutral detergent fiber (“RUNDF”) content of the feed components. The RUNDF content being a fiber portion in the feed ration orally ingested and forming a mat of undegraded fiber in the rumen. The feed ration is formulated based on the received RUP and RUNDF values for the feed components.

In yet another implementation, a method of determining a RUP level in a feed ration involves generating a calibration curve of RUP content in feed components used in feed rations based on previously analyzed feed component samples; analyzing feed components for RUP content; and determining a RUP content in the feed ration containing the feed components based on comparing the calibration curve with results of the analyzed feed components.

### BRIEF DESCRIPTION OF THE DRAWINGS

FIG. 1 shows a graph of dietary components that may cause variations in fat corrected milk production in lactating dairy cows.

FIG. 2 shows a graph listing various protein sources and their related degradation rates and RUP levels.

FIG. 3A is a flowchart of a method for improving milk production and/or reducing the cost of milk production by manipulating RUP in the feed ration, according to the present disclosure.

FIG. 3B is a flowchart of a method for determining RUP content in a feed ration, according to the present disclosure.

FIG. 3C is a flowchart of another method for improving milk production and/or reducing the cost of milk production by manipulating RUP in the feed ration, according to the present disclosure.

FIG. 4 shows a schematic of a feed ration formulated for a ruminant according to the present disclosure, which provides RUP levels to support normal milk production.

FIG. 5 shows a schematic of a feed ration formulated for a ruminant according to the present disclosure to provide RUP levels to support normal milk production in which the RUP has a slower rate of passage through the rumen.

FIG. 6 shows a schematic of a feed ration formulated for a ruminant according to the present disclosure to provide RUP levels to support increased milk production in which the RUP has a higher rate of passage through the rumen.

FIG. 7 shows a schematic of a feed ration formulated for a ruminant according to the present disclosure, used to reduce feed costs by substituting high cost feed ingredients with low cost feed ingredients.

### DETAILED DESCRIPTION

Variations to nutritional content in animal dietary components including protein (e.g., alfalfa hay, soybean meal), forage (e.g., corn silage, alfalfa hay, wheat straw, and the like), starch, and byproducts, as well as non-feed components, may cause variations in animal performance including milk production, dry matter intake, milk component yields, energy storage and feed efficiency. For example, FIG. 1 shows dietary components account for variations in fat corrected milk production in lactating dairy cows, and forage, starch, protein and byproducts, together account for about 65% of variation in fat corrected milk in lactating dairy cows. If not controlled, this variation may compromise animal performance such as milk and milk component production, as well as dry matter intake and feed efficiency. To efficiently and cost-effectively feed animals such as livestock animals, including ruminants, these sources of variation should be measured and controlled in order to predict animal performance.

In addition, the cost of feed components affects the cost of milk production. Protein sources are generally known as the most expensive feed ration component, and some protein sources such as blood meal may add a significant cost to milk production. FIG. 2 illustrates several protein sources and their associated protein degradation rates and RUP levels. However, for each protein source, the actual degradation rate may vary from the levels depicted and thus the RUP levels may vary. This variation may be due to differences in the chemical and structural nature of protein fractions. Because the amount of crude protein and RUP in protein rich feed components is generally given a fixed value, and the fixed protein content values are typically used in formulating feed rations, the producer may incur added expense to milk production or may not reach optimum ruminant performance when values used to formulate the feed ration are not accurate. For example, when the actual protein and RUP content in protein sources varies from the predicted or fixed amounts, using the predicted or fixed protein values in calculating the feed ration may result in decreased milk production when the actual protein and RUP content is low, or may result in an increased cost of milk production when the actual protein and RUP content is high. In addition, excess protein levels in the diet may result in increased ammonia waste in urine, which may be problematic for the environment.

Embodiments herein provide systems and methods for accurately determining RUP in protein sources and using the RUP levels to calculate feed formulations, thereby enabling the efficient use of dietary protein in feed rations. Accurate determination of RUP levels and their constituent rumen undegraded amino acid levels (“RUAA”) enables metabolizable protein (“MP”) and its constituent metabolizable amino acid levels (“MAA”) to be more accurately predicted, which may enable the nutritionist to formulate feed rations to improve animal performance (e.g., increase milk production and/or meat production, improve feed to gain efficiency, and/or optimize feed intake), decrease feeding costs or both. RUAA content is the constituent amino acids in RUP, and calculation of RUAA involves multiplying the proportion of each constituent amino acid in each ingredient by the RUP percent to arrive at a rumen undegraded value for each amino acid. Alternatively, RUAA content could be determined from direct methods of detection or predicted from previously developed equations relating RUAA to spectral data. RUAA would include known amino acids, such as methionine, lysine, histidine, threonine, leucine, arginine and others. In one example, RUAA content may be determined for methionine and lysine and may be used in formulating feed rations, or its nutritional constituents such as crude protein.

FIG. 3A is a flowchart of a method 100 for formulating a feed ration for improving performance and/or reducing feed costs by manipulating the RUP in a feed ration. The method 100 involves sampling feed sources from a producer's farm or from the producer's available feed sources in operation 110. The samples are processed, generally in a lab, and analyzed for nutritional component composition including RUP content in operation 120. Results of the analysis are used to formulate or reformulate a feed ration in operation 130. After feeding the formulated or reformulated feed, animal performance, such as milk production levels, may be monitored in operation 140. In some aspects, the monitoring information is provided for performing milk composition diagnostics. One or more steps in method 100 may be periodically repeated, in part, due to the results of the monitored animal performance, or changes in the digestibility or protein content of the forage and grains and therefore in the RUP content of the ingredients, or all of these.

In operation 110, the primary feed sources available for use in a feed ration such as feed components containing protein, fiber, starch, or all of these, may be sampled. Examples of protein sources may include: soybean meal, which may be dehulled; cottonseed meal; corn gluten meal; canola meal; and alfalfa hay. Additional exemplary protein sources include plasma protein, egg protein and by-product protein. Animal by-product meals such as meat meal, poultry meal, blood meal, feather meal and fish meal may also be included. Plant by-product meals such as wheat middlings, soybean hulls and corn by-products may additionally be included. Microbial proteins such as torula yeast and brewer's yeast, as well as distillers grains, brewers grains, fine ground corn and gluten feed, may also be sampled. Examples of fiber sources may include forage, such as: alfalfa silage, corn silage, wheat silage, sorghum silage, oat silage, grass silage, ryegrass silage, barley silage, triticale silage, grass hay, alfalfa hay, oat hay, wheat hay, barley hay, ryegrass hay, triticale hay, oat straw, wheat straw, barley straw, whole cottonseed, cottonseed hulls, beet pulp or any combination thereof. Examples of starch may include grains such as: corn grain, corn silage, corn gluten feed, corn germ meal, corn starch, corn byproducts, sorghum grain, sorghum silage, sorghum byproducts, milo, wheat grain, wheat silage, wheat bran, red dog wheat, wheat germ, wheat flour, wheat middlings, wheat byproducts, barley grain, barley silage, barley byproducts, oat grain, oat silage, oat byproducts, bakery byproducts, hominy feed, peas, brewers grains, distillers grains, malt sprouts, rice, rice bran, rice flour, rice byproducts, cereal feed, sucrose, lactose, glucose, dextrose, maltose, cassava, potatoes or other starchy tubers or any combination thereof. As may be appreciated, the protein, fiber and starch sources described above may also provide dietary sources of multiple nutritional components. Thus, for example, alfalfa hay may provide a source of protein and fiber.

In operation 120, the sampled protein sources are analyzed to determine degradation values, such as ruminal degradation values, in order to determine their rumen undegraded value by difference, or the undegraded values can be directly determined. The samples may be analyzed for RUP content, rumen undegraded amino acid (RUAA) content, or both, one or both of which may be used to predict MP and metabolizable amino acids. The samples may additionally or alternatively be analyzed for rumen undegraded neutral detergent fiber (RUNDF) content or for the content of other nutritional components such as starch. Based on the analysis, the nutritional content may be expressed in relation to the dry matter in a feed ration, in relation to one or more nutrients in the feed ration, or both. For example, a level of RUP may be expressed in relation to dry matter, in relation to a crude protein level, or both.

In one example, the samples may be subjected to in vivo, in situ or in vitro analysis where degradation of the nutritional component over time is determined. For example, using an in vitro analysis for operation 120, the starting protein content for the ingredients analyzed may be measured by chemical and/or enzymatic methods; and the in vitro microbial or enzymatically digested residue amounts are compared with the initial amounts to calculate RUP, for example, as a percent of their original amounts. However, other analysis methodologies may also be used in connection with operation 120, including spectrophotometric methods (IR and/or NIR analysis) or other rapid testing methods that provide results within 24 to 48 hours from the time the sample reaches a lab (e.g., a lab remote from the sample source or a lab located at the producer's location). For example, samples may be subjected to NIR analysis and may be compared to degradation results of reference samples previously subjected to one or more NIR, in vitro, in vivo or in situ analyses, and a degradation value may be calculated based thereon. In particular, a RUP content in a feed ration may be determined according to the method 122 of FIG. 3B in which a calibration curve of RUP content in feed components is generated based on previously analyzed feed component samples in operation 124. One or more feed components from a feed ration are analyzed for RUP content in operation 126. For example, ruminal degradation rates of the protein and the rate of passage of the protein may be used to calculate RUP content. In some aspects ruminal degradation may be expressed as:

Digestibility=Potential Digestible fraction×[k_{d}/(k_{d}+kₚ)], where k_{d} is the rate of digestion and kₚ is the rate of passage. Based on a comparison of the results of the analysis with the calibration curve, a RUP content in the feed ration is determined in operation 128.

In some implementations, the analysis of operation 120 may yield degradation values that may be converted into an indexing system that has a linear relationship to the in vivo degradability and undegraded portion of the feed component. For example, for protein sources, the analysis may yield protein degradation values for the in vivo degradability as well as the RUP content of the protein source, which may be used to generate a RUPN index rating that may include values that range from 1 to 11, where 1 is representative of a protein source of low or slow ruminal digestion and therefore high RUP (e.g., blood meal) and 11 is representative of a protein source of high or fast ruminal degradation and therefore a low RUP (e.g., alfalfa hay). For fiber sources, the degradation values may be converted for use in the FPN indexing system for rating fiber, which includes an index with values ranging from 60 to 200, where 60 is representative of an NDF source of low or slow ruminal digestion (e.g., cottonseed hulls) and 200 is representative of an NDF source of high or fast ruminal digestion (e.g., wheat silage).

In addition, the degradation values may be converted into a degradation constant, which may be the rate of ruminal degradation of the nutritional source per hour. For example, a protein degradation rate constant value of 0.08/hr for the protein in soybean meal composed of 50 percent protein means that the insoluble protein degrades or digests in the rumen at approximately 8 percent per hour. Generally, each protein source is assigned its own degradation rate (k_{d}) ranging from 0.01 to 0.14/hr. While the insoluble fraction of the protein source may be assigned the protein degradation rate constant, the soluble content of a protein source is generally considered to be completely dissolved in the rumen and therefore is assigned a protein degradation rate constant of an extremely high value, such as 100 percent per hour. Thus, for soybean meal, 80 percent of the protein may be insoluble in the rumen and have a protein degradation rate constant of 0.08/hr, and the remaining 20 percent of the protein may be completely soluble and degradable in the rumen.

In operation 130, the results of the analysis (e.g., spectral results, in situ or in vitro testing results that may be converted into RUP percent or an indexing number) for some or all of the dietary components may be used to formulate or reformulate a feed ration or portions thereof (e.g., dietary components such as crude protein, fiber and/or protein). For example, the analysis results may be provided as input to a feed formulation calculator along with the feeding rates of each dietary component. Using a feed formulation calculator, the amount of the protein sources in the feed, such as soybean meal and alfalfa hay, may be provided as input along with their associated CP values, soluble protein values, RUP values, RUAA values, RUPN values, RUAAN values, protein degradation rate constants, feeding rates, and/or ruminal passage rates.

In some implementations, in operation 130, the metabolizable protein and amino acid levels in the diet may be calculated based on the RUP for each feed component; the pounds of protein source in the feed formulation is multiplied by the protein amount (% of protein source) as well as by the RUP or the RUAA fractions thereof. For example, for alfalfa hay, the number of pounds of alfalfa hay is multiplied by the percentage of protein and by the RUP values, which may be determined based on relationships obtained from previously analyzed samples using the analysis methods described above.

In some implementations, after the conversion of the RUPN results to RUP values as a percent of protein based on a linear scale, the formulation calculator sums the contributions of the nutrient fractions from all ingredients (e.g., all sampled ingredients). In other instances, the RUP fraction can be measured directly, such as by measuring residue amounts after microbial digestion in ruminal fluid, and expressed as a percent of ingredient DM, without measuring the crude protein content of the undigested ingredient.

Various feed formulation calculators may be used in operation 130 to formulate or reformulate a feed ration and are not limited to a particular type of feed formulation calculator. For example, these RUP and RUAA values can be used in feed formulation calculators such as the 2001 NRC Nutrient Requirements of Dairy Cattle or the Cornell Net Carbohydrate and Protein System or the Dynamic Nutrition System formulation model. Due to the accurate RUP and RUAA levels in the protein sources determined by aspects of method 100, the feed formulation calculators may provide more precise values for the amount of the protein sources to include in the feed ration for improved performance or cost savings. However, in some aspects, where a feed formulation calculator is used to calculate an initial feed formulation, e.g., a baseline feed ration, the same calculator may be used in reformulating the feed. However, the use of multiple feed formulation calculators may be used in order to arrive at a feed ration acceptable for a producer's stock.

In some aspects, in operation 130, analysis results from operation 120 may be compared with other RUP information and target levels based on animal production results (e.g., milk production) and the feed rations may be formulated or reformulated. For example, the summed amounts of the pounds of RUP may be compared to the dietary targets for either RUP or MP, and the feed component amounts are adjusted or reformulated to the dietary targets for improving dry matter intake, increasing feed efficiency, increasing yield of milk components or combinations.

In operation 140, animal performance such as milk production levels are monitored after feeding the formulated or reformulated feed to the lactating dairy cow. For example, milk composition diagnostics may be performed in connection with the monitored milk production levels. In response to the monitored animal performance, if the ruminant's performance is not at a desired level, then method 100 may return to operation 130 for reformulation or to operation 110 for sampling feed components. The feed ration may be reformulated to increase milk production and/or to increase milk components, and/or increase dry matter intake, and/or increase feed efficiency.

This process is periodically repeated, in part, due to the changes in the RUP content of protein sources. The process may be repeated every 2-4 weeks, 1-2 weeks, 4 weeks, and so on, in order to account for differences in RUP in the feed components over time. Particularly, RUP may change as a result of environmental effects during growth, processing effects (such as heat treatment of byproducts during drying) and physicochemical differences among, protein types and sources.

FIG. 3C shows another method 200 for improving performance of a ruminant and/or reducing feed costs by formulating a feed ration using RUP calculations, which may be used by a nutritionist or a producer. According to FIG. 3C, a level of RUP in relation to dry matter in a ruminant feed ration or in relation to the crude protein level in a ruminant feed ration is received 210; the feed ration is modified 220 based on the received RUP level; and the modified feed ration is fed 230 to the ruminant to improve performance and/or reduce feed costs.

In operation 210, the received RUP level is based on an analysis of RUP present in one or more feed components in the feed ration, such as feed components contributing crude protein to the ration

. The analysis methods used to determine the RUP level may be any of the chemical, enzymatic, microbial degradation, and/or spectral analysis methods described above in connection with the method 100 of FIG. 3A. In some aspects, operation 210 may additionally involve receiving RUNDF levels based on an analysis of the feed components as discussed above in connection with method 100.

In operation 220, the modified feed ration utilizes the received RUP level to control the amount of RUP exiting the rumen. In some aspects, these modifications may adjust a rate of passage and/or an extent of passage of the protein through the rumen in the undegraded state. In addition or alternatively, the modification may involve selecting combinations of ingredients of various RUP levels to achieve the desired dietary RUP amount.

In some implementations, decreasing crude protein levels or increasing RUNDF levels may result in a decreased rate of passage of the RUP, while increasing the protein levels or decreasing RUNDF may increase the rate of passage of the RUP. In a particular example, with a modified rate of passage due to a change in RUNDF level, the level of RUP entering the small intestine may change even when the level of crude protein remains constant in the diet (see e.g., FIGS. 4-6). In another example, a level of crude protein and/or RUNDF in relation to the dry matter in the feed ration may be selected and the other of the protein and RUNDF may be adjusted, thereby modifying the rate or extent of passage of the RUP to facilitate reaching a target level of RUP. In yet another example, an extent of passage of the protein through the rumen may be modified in the feed ration by processing the protein to modify a degradation rate.

Operation 230 involves feeding the modified feed ration to the ruminant using traditional feeding methods, for example, in order to improve performance and/or reduce costs. In yet a further example, utilizing known RUP levels of available feed components, a combination may be selected to achieve the desired dietary RUP amount. Moreover, known RUNDF levels of the available feed components may be used in combination RUP-containing feed component to achieve the desired effect.

In certain implementations, the methods of the present disclosure may be used to obtain target protein intake, digestibility, RUP and MP levels for normal milk production or increased milk production.

In one implementation, the methods may be used to formulate a feed ration using calculated protein sources including RUP and/or MP and/or RUAA levels as well as fiber and starch levels to support a normal milk production with a normal feed intake, normal feed efficiency and normal digestibility. For example, RUNDF levels in the feed ration may promote a fiber mat (composed of fiber, including NDF, alfalfa hay, wheat straw and so on) of a normal size in the rumen, which may enable the protein sources to pass through the rumen at a normal rate in order to maintain a normal amount of RUP, RUAA, MP and milk production. A normal passage rate of the protein sources may occur at the protein degradation rate constant discussed above (e.g., 0.08/hr for soybean meal), and normal milk production is understood as a range of milk production at a given point in the lactation cycle of a lactating dairy cow. A targeted level of RUNDF and RUP represents the combination of rate of passage and rate of protein degradation to provide an amount of RUP to support normal milk production with acceptable feed efficiency. For example, normal levels of 3.5% fat corrected milk production from 3 to 12 weeks of lactation are expected to be in a range of 70 to 100 lbs. per day. Because the ruminal digestibility and RUP of each protein component is known to a higher level of accuracy compared to prior approaches, specifically, due to the analysis methods described above in connection with method 100, the ratio of the components fed to the cow may be manipulated to consistently achieve normal milk production.

FIG. 4 shows a schematic of a feed ration formulated according to the present disclosure, which provides RUP and RUNDF levels to support a normal milk production in a ruminant 400. According to FIG. 4, the ruminant diet is optimized for normal RUP and DM intake, digestibility, feed efficiency and production. For example, the feed ration may include a crude protein amount of 10 lbs. per head per day with a RUP level of 4.5 lbs. and microbial protein level of 4.5 lbs. per head per day; along with normal levels of RUNDF in the rumen to promote normal digestibility, which may be fed to the cow to promote retention of the protein 410 in the rumen 405 for a normal period of time. The intake, digestibility, feed efficiency and production rates are, in part, based on the normal sized fiber mat 420 (e.g., RUNDF and other fiber components) within the rumen of the ruminant, which promotes passage of RUP through the rumen to result in a desired level of RUP reaching the small intestine to sustain normal milk production levels. Although the fiber mat 420 within the rumen eventually moves out of the rumen and is excreted, the methods of the present disclosure take into account the feeding rate of the cow and therefore calculates the amount of RUNDF and other fiber components in the rumen at a given time based on the feeding rate of all or substantially all forage ingredients in the diet. For example, feeding 10 lbs. of DM from alfalfa hay containing 40 percent NDF with 60 percent rumen undigested NDF contributes 2.4 lbs of RUNDF to the total diet RUNDF amount. In some implementations, a normal RUNDF level may be in the approximate range of 8 to 13 weight percent in relation to the dry matter of the feed. However, the recommended RUNDF level may be determined as a portion of the fiber recommendation.

In some implementations, the methods of the present disclosure may be used to increase the feed efficiency of the ruminant but at a similar milk production and lower dry matter intake by decreasing the rate of passage of the feed ration components through the rumen 405. To account for the lower rate of passage, the amount of RUP may be increased in the diet to account for the additional residence time and therefore degradation of the RUP in the rumen 405. For example, the RUP may be increased to a level so that the same amount of RUP reaches the small intestine as compared to a feeding ration formulated for a normal rate of passage as described above in connection with FIG. 4.

FIG. 5 shows a schematic of a feed ration formulated for a ruminant 400, which provides RUP and RUNDF levels for optimizing the diet to achieve a higher feed efficiency and higher digestibility with normal milk production. The feed ration may be formulated with elevated levels of fiber-containing feed components to promote a larger fiber mat in the rumen resulting in retention of the starch and protein components in the rumen for a relatively longer time compared to a feed ration formulated for a normal rate of passage. According to FIG. 5, the ruminant 400 diet may be formulated with a crude protein level of 10 lbs. per head per day and an elevated RUNDF level in order to yield a RUP of 4.0 lbs per day and a microbial protein production level of 5.0 lbs. per day with a normal level of milk production due to the elevated level of degradation of protein in the rumen.

To illustrate, comparing the feed rations of FIGS. 4 and 5, each may contain the same amount of crude protein in the diet, and with respect to FIG. 4, a normal amount of RUP may account for 6 to 11 weight percent in relation to the dry matter of the feed; and a RUNDF level present in the rumen may be in the approximate range of 8 to 13 weight percent in relation to the dry matter of the feed. In the feed ration implementation of FIG. 5, the RUP may account for 5 to 10 weight percent in relation to the dry matter of the feed, while the RUNDF level present in the rumen may be in the approximate range of 10 to 15 weight percent in relation to the dry matter of the feed, or at an increased level compared to the normal amount of fiber or RUNDF.

In the example of FIG. 5, the level of protein may be maintained at normal intake levels but degraded to a larger extent in the rumen and therefore a smaller amount of RUP is available to the small intestine, however, without decreased milk production, e.g., due to the production of microbial amino acids synthesized from the degradation of the protein in the rumen. In alternative implementations, the level of protein may be increased so that the RUP level reaching the small intestine is elevated to provide amino acids to the small intestine and to maintain milk production levels at a normal rate or an increased rate. In this example, calculation of the feed ration may take into account the rate of ingestion of the RUP and may adjust the RUP amounts (e.g., weight) to account for the decreased rate of passage and therefore the increased rate of degradation in the rumen. In addition or alternatively, calculation of the feed ration may adjust the inherent RUP value (e.g., the RUPN value) of the protein source to account for the decreased rate of passage and, for example, the calculation may assign an actual RUP value or an actual RUPN value based on the decreased rate of passage, which may be a value that is smaller relative to the inherent RUP and RUPN value.

The feed ration example of FIG. 5 may be useful where the producer has limited resources (e.g., feed components such as corn grain) available and desires to substitute with protein sources or other feed ingredients in the feed ration. For example, the region in which the producer is situated may affect the availability of certain resources. In the Southwest region of the US, corn grain may not be readily available or may be difficult to grow. Accordingly, the available resources may be evaluated to determine a level of protein and RUP that achieves normal milk production, for example, at a reduced starch level and increased RUNDF level.

FIG. 6 shows a feed ration formulated according to the present disclosure, in which the ruminant diet is optimized for higher intake and milk production, but at a higher feed cost and lower feed efficiency. According to FIG. 6, the crude protein intake may be about 10 lbs. per head per day with a lower RUNDF resulting in a higher feed intake. As a result, the ruminant experiences less rumen fill, a faster rate of passage of the nutritional components including protein and lower overall digestibility, which provides a RUP level of 5.0 lbs. per head per day and a microbial protein level of 4.5 lbs. per head per day to yield the increased milk production.

With further reference to FIG. 6, the fiber level in the feed ration may be relatively low so that a smaller fiber mat 420 promotes movement of and an increased level of the RUP component through the rumen 405, while at the same time increasing the supply of amino acids to the small intestine. For example, compared to the normal amounts of RUP (e.g., 6 to 11 weight percent in relation to dry matter of feed) and normal amounts of RUNDF (e.g., 8 to 13 weight percent in relation to dry matter of feed), in the example of FIG. 6, the RUP may be 7 to 12 weight percent in relation to the dry matter of the feed, while the forage RUNDF may be in the approximate range of 6 to 11 weight percent of ruminally undigested forage NDF component in relation to the dry matter of the feed.

In this example, calculation of the feed ration may take into account the rate of ingestion of the RUP and may adjust the RUP amounts (e.g., by weight) in the ration to account for the increased rate of passage. In addition or alternatively, calculation of the feed ration may adjust the inherent RUP value (e.g., the RUPN value) of the protein source to account for the increased rate of passage and, for example, the calculation may assign an actual RUP value based on the increased rate of passage, which may be a value that is larger relative to the inherent RUP value.

Further, as explained above, because the methods take into account the feeding rate of the cow, in the feed ration example of FIG. 6, the amount of RUNDF and other fiber components in the rumen at a given time may be lowered based on the feeding rate. This lowered fiber level in comparison to the DM intake level may also be expressed as a RUNDF level, or the RUNDF level may be taken into account as part of the fiber recommendation in the fiber and starch feed levels or amounts.

The feed ration example of FIG. 6 may be useful where the producer has a small amount of protein containing feed components available and/or a small amount of fiber containing components available. In addition or alternatively, the producer may target increased milk production, which may cause an increase in feed costs, for example, in situations where the market for feed is low (e.g., low priced) and/or the market for milk is high (e.g., increased payments to producer per lb. of fat corrected milk).

Although the preceding examples of FIGS. 4-6 provide RUP recommendations according to RUNDF levels, in some feed rations, the RUP content may be 6 to 11 percent for any RUNDF level. Particularly, the RUNDF level may be selected with the aim to reduce cost, and based on market conditions, the low, normal or high RUNDF levels may be selected.

In another implementation, the methods may be utilized by nutritionists and producers to reduce the feed costs while maintaining milk production at a desired level. FIG. 7 shows a feed ration formulated according to the present disclosure, in which the ruminant diet is optimized to reduce feed costs by substituting high cost feed ingredients (e.g., higher cost proteins, starch and/or fiber components) with low cost feed ingredients (such as lower cost proteins, silage or alfalfa hay), while providing a ruminant diet optimized for normal RUP and DM intakes, digestibility, feed efficiency and production, according to the present disclosure. In this implementation, any of the feeding systems described above may be used to maintain milk production levels or improve milk production, while at the same time reducing feed costs from a starting feed ration. According to FIG. 7, the crude protein intake may be 10-10.5 lbs. per day of lower cost protein sources and the RUNDF level may be at a normal level for normal intake in order to cause the ruminant to experience a normal rumen fill and rate of passage for a RUP of 4.5 lbs. per head per day and a microbial protein level of 4.5 lbs. per head per day for a normal milk production.

Accordingly, the feed ration formulated according to FIG. 7 may be useful where the producer or nutritionist has achieved a normal milk production and desires to reduce feed costs while maintaining normal milk production. In this case, a feed ration calculator may recommend the same or similar protein amounts, but may recommend different, less expensive, feed components (e.g., recommend canola meal instead of protected soybean meal).

As may be appreciated, a number of methods may manipulate protein and RUP in the ruminant diet by adjusting the rate of degradation and/or rate of passage of the protein components to achieve various outcomes. It will be appreciated that improved performance or normal performance achieved by using the feed formulation methods and RUP determination methods of the present disclosure may be relative to prior performance levels, which may be lower than expected. Thus, a normal milk production in a ruminant ingesting feeds formulated according to the methods herein may be an improvement, particularly where the prior performance of the ruminant is lower than expected.

Each of the examples above may further involve analyzing milk from the ruminant for milk fat content and milk protein content, and increasing the amount of the RUP if the milk protein content is low and decreasing the RUP if the milk protein content is high.

In another example, once the inherent degradation rate or amount of the RUP portion of the protein source is determined, e.g., through NIR analysis of the protein components, the protein source may be processed to achieve a selected RUP level and/or RUAA level. For example, the protein source may be processed by grinding, chopping, steam flaking, steam rolling, extrusion and/or chemically or physically treating the protein to modify its rate of ruminal digestion and therefore its RUP. Chemically or physically treating the protein components may include but is not limited to aldehyde treatment, treatment with bases or acids, alkaline peroxide, heat treatments, resins, binders or coatings. Genetically modifying crops to reduce protein degradation in the rumen (e.g., higher tannin levels in alfalfa) can also be used to improve RUP value. By exposing more surface area of the protein source, e.g., by grinding or chopping the protein source, the RUP value may be lowered from the inherent degradation amount of the RUP portion of the protein source. Protecting the protein source from ruminal degradation by chemical treatment, for example, may raise the RUP value of the protein source. Once the selected RUP level has been reached, the adjusted RUP level or RUPN rating and/or RUAA level or RUAAN rating for the protein source may be used as input for the feed ration calculator described above to determine the amount of the protein source needed to yield the desired milk production outcome.

The embodiments provided herein may benefit the dairy producer where ingredient cost savings is desirable by substituting high cost ingredients such as protected soybean meal with lower cost ingredients such as alfalfa hay, while not affecting milk production, e.g., while maintaining desired milk production levels. This may also provide the producer with informed or improved economic evaluations of ratio of feed costs to milk production costs.

The dairy producer having limited protein resources may benefit by adjusting the diet to a low protein intake in combination with a high rate of passage of the protein (e.g., a lower fiber content), while not adversely affecting milk production.

The embodiments provided herein may also be useful in crop planting planning because hybrid selection and harvest and feed storage management may be adjusted based on the calculated values. This also enables the producer to effectively manage their feed inventory.

Methods for adjusting RUP in diets may be applicable to a number of different types of animals, and thus the methods provided herein are not limited to cows and other ruminants may benefit from the methods provided herein.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

In view of the foregoing, aspects of the methods of the present disclosure may be implemented using a computer or computer components such as a processor and a memory. For example, analysis of the feed component samples may be performed using a computerized analysis device, and the results may be used as input in a feed formulation calculator for formulating or reformulating a feed ration. The feed formulation calculator may be embodied in software and/or hardware, and a computer processor may execute instructions for receiving and analyzing the input and for reformulating the feed ration based on the received and analyzed data.

Accordingly, aspects of the present disclosure may be provided as a computer program product, or software, that may include a data storage units provided as non-transitory machine-readable medium having stored thereon instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (ROM); random access memory (RAM); erasable programmable memory (e.g., EPROM and EEPROM); flash memory; and so on. Accordingly, the methods provided herein may be implemented on a computer system communicatively coupled to other computer systems, and/or on a communicatively coupled network of computers, having processing units, memory storage units, communications units, and communication links. The processing units retrieve and execute commands stored in the memory storage units and provide output in the form of a delivered message or delivering output to a communicatively coupled display.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them, and variations, modifications, additions, and improvements are possible. More generally, embodiments in accordance with the present disclosure have been described in the context or particular embodiments. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A method for improving performance of a ruminant and/or reducing feed costs, the method comprising:
receiving a level of rumen undegraded protein (“RUP”) in relation to dry matter in a ruminant feed ration, the RUP level comprising a level of protein in the feed ration to be orally ingested and passed through the rumen in an undegraded or undigested state, wherein the RUP level is based on an analysis of RUP present in one or more feed components in the feed ration;
modifying the feed ration based on the received RUP level to control an amount of RUP exiting the rumen; and
feeding the modified feed ration to the ruminant.

2. The method of claim 1, wherein modifying the feed ration comprises adjusting a level of crude protein in relation to the dry matter in the feed ration.

3. The method of claim 1, further comprising:
receiving a level of rumen undegraded neutral detergent fiber (“RUNDF”) in relation to the dry matter of the feed ration; and
wherein modifying the feed ration comprises adjusting the level of RUNDF to modify a rate of passage of the protein through the rumen.

4. The method of claim 1, wherein the analysis of the RUP comprises at least one of chemical degradation analysis, enzymatic degradation analysis, microbial degradation analysis or spectral analysis.

5. The method of claim 1, wherein modifying comprises at least one of adjusting a rate of passage or an extent of degradation of the protein in the feed ration through the rumen or by selecting combinations of ingredients of various RUP levels to achieve the desired dietary RUP amount.

6. The method of claim 5, wherein adjusting the extent of degradation of the protein in the rumen comprises processing the protein to modify a ruminal degradation rate by at least one of exposing protein surface area to increase the ruminal degradation rate or protecting the protein surface area to decrease the ruminal degradation rate.

7. The method of claim 1, wherein modifying further comprises modifying the feed ration based on a measured performance of the ruminant.

8. The method of claim 7, wherein the measured performance comprises at least one of milk production or meat production.

9. A method for formulating a ruminant feed:
selecting feed components for use in a ruminant feed ration;
receiving nutritional composition analysis results of the selected feed components, said results comprising a rumen undegraded protein (“RUP”) content and a rumen undegraded neutral detergent fiber (“RUNDF”) content of the feed components, the RUP content comprising a protein content in the feed ration to be orally ingested and passed through the rumen in an undegraded or undigested state and the RUNDF content comprising a fiber content in the feed ration orally ingested and forming a mat or mass of undegraded fiber in the rumen; and
formulating the feed ration based on the RUP and RUNDF content of the feed components.

10. The method of claim 9, wherein formulating the feed ration comprises selecting a level of RUNDF in the feed ration thereby modifying a rate of passage of the protein content in the feed ration through the rumen.

11. The method of claim 10, wherein formulating the feed ration further comprises adjusting a level of crude protein in relation to the dry matter in the feed ration to reach a selected RUP content.

12. The method of claim 9, wherein formulating the feed ration comprises adjusting an extent of degradation of the protein in the rumen by processing the protein to modify a degradation rate.

13. The method of claim 9, wherein the feed ration is formulated to select at least one of a RUP level or a RUNDF level to improve ruminant performance or to reduce feed costs.

14. The method of claim 9, further comprising:
analyzing animal performance after feeding the formulated feed ration; and
reformulating the feed ration based on the analysis, wherein reformulating comprises providing 6-11 percent RUP in relation to dry matter of the feed.

15. A method of determining a rumen undegraded protein (“RUP”) level in a feed ration, the RUP level comprising a level of protein in the feed ration to be orally ingested and passed through the rumen in an undegraded state, the method comprising:
generating a calibration curve of RUP content in feed components used in feed rations based on previously analyzed feed component samples;
analyzing one or more feed components for a RUP content; and
determining a RUP content in a feed ration containing the one or more feed components based on a comparison of results of the analyzed feed components with the calibration curve.

16. The method of claim 15, wherein analyzing comprises analyzing through rapid testing.

17. The method of claim 16, wherein rapid testing comprises using near infrared reflectance spectroscopy.

18. The method of claim 16, wherein rapid testing comprises utilizing testing methods providing results within 24 to 48 hours.

19. The method of claim 15, wherein the previously analyzed feed component samples are analyzed by at least one of in vitro analysis, in situ analysis or in vivo analysis.

20. The method of claim 15, further comprising determining a rumen undegraded amino acid (“RUAA”) content in the feed ration, wherein the RUAA content comprises constituent amino acids in the RUP determined by calculating the proportion of each constituent amino acid in the RUP.
